SEQUENCE LISTING (9 sequences)

SEQ ID NO: 1 - DNA, 960 bp, Homo Sapiens
agtaggggcccgggcggaggcggtggcgggatggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcaggtgagctggaggtcggggagggggcctggctgccgggaacccgggcctgggcgggacg

SEQ ID NO: 2 - DNA, 261 bp, Homo Sapiens
gatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtgga

SEQ ID NO: 3 - DNA, 873 bp, Homo sapiens
atggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcagg

SEQ ID NO: 4 - DNA, 263 bp, Homo sapiens
gatcaaggtggagttcgaggagctgctgcagaccaagacggcctttttttttttggaggggctgagcctgcgcgacgtgttcctgggtgacaccgtgccctacatcaagaccatccggctggtgcggcccgtggtggcttcgggcaccggcgagcccgacgaacccgatggggacgctctgcccgccacctgcccgggggagctggcctttgaggcggaggtggagtacaacggcggcttccacctggccatcgacgtggatc

SEQ ID NO: 5 - DNA, 22 bp, Artificial Sequence
  Synthetic Primer
aaggtggagttcgaggagctgc

SEQ ID NO: 6 - DNA, 22 bp, Artificial Sequence
  Synthetic Primer
gtggaagccgccgttgtactcc

SEQ ID NO: 7 - DNA, 3832 bp, Homo sapiens
  CDS (178)..(3639)
ccgctgcgggctcgggcgccgcagcgcgccggcccgagcccctggacgaggcccacggagccgctcgccccgacccagccgcccgatgtcctcaaaatggaggcagcgggggcggcggcgtgaagaaagcggcgctgtgggcgcgggagtaggggcccgggcggaggcggtggcgggatggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcaggtttaagccgttttttccataccagaccttgcaaggatttgaagaagatgaagagcatatccatatacaacaatgggcacttactgaaggccgtcttaaagttacgttgttagaatgtagcaggttactcatttttggatcctatgacagagaggcaaatgttcattgcacacttgagttaagcagtagtgtttgggaagaaaaacagaggagttctattaagacggttgaattaataaaaggaaatttacaaagtgttggacttacacttcgtcttgtccagtcaactgatgggtatgctgggcacgtcatcattgaaactgtggctccaaactcgcctgctgcaattgcagatcttcagcggggagatcgacttatcgccattggaggtgtgaaaatcacatcaacactgcaagtgttgaagcttatcaagcaggctggtgaccgagtcctggtgtactatgaaaggcctgttggccagagtaatcaaggtgcagtgctgcaagataactttggccagttggaagaaaactttttgtcaagctcatgccaatcgggttatgaagaggaagctgccgggttgacagtagatactgaaagtagagagctggattctgaatttgaagacttggcaagtgatgtcagagcacaaaatgagttcaaagatgaggcacaatcattaagtcatagtcccaaacgtgttccaacaacactttctattaaaccccttggagctatatcaccagttttaaaccgtaaattagctgtaggaagtcacccactaccaccgaaaattcagtccaaagatggaaataaacctccacccctaaaaacttctgagataacagacccagcacaagtgtcaaaaccaacccaaggatctgctttcaaaccacctgtgccaccacgaccacaagcgaaagttcctttgccttccgccgatgctccaaatcaggcagaaccagatgttctcgttgaaaagccagagaaggtggtgccacctcctcttgtagataaatctgctgaaaagcaagcaaaaaatgtggatgccatagacgatgcagctgcacctaagcaatttttagcaaagcaagaagtggccaaagatgtcacttcagaaacttcctgccctactaaggacagttcggacgaccgtcaaacatgggaatcatcagaaattctttatcgtaataagctaggaaaatggacaagaaccagagcatcctgtttgtttgacatagaagcctgtcacaggtacttaaacattgcattgtggtgcagggatcctttcaagttgggaggtctcatctgtttggggcatgttagtttaaaacttgaagatgtggctttaggatgcctagctacatcaaacacggaatacctttccaaattgagactggaagccccctcacctaaggctatagtcactagaaccgcactacgcaatctgagtatgcaaaagggattcaatgacaaattttgctatggtgacattactattcacttcaaatatttgaaagaaggagaatcagaccaccatgtagttactaacgtagaaaaagaaaaagaaccccatttggttgaagaagtttctgttctccctaaagaggagcaatttgttggacagatgggtttaacagaaaacaaacacagttttcaggatactcagttccagaacccaacatggtgtgactactgtaagaaaaaagtttggactaaagcagcttcccagtgtatgttttgtgcttatgtttgccataaaaaatgtcaagaaaagtgtctagctgagacttctgtttgtggagcaactgataggcgaatagacaggacactgaaaaaccttaggctggaaggacaggaaaccctcttaggcctgcctcctcgtgttgatgctgaagctagcaagtcagtcaataaaacaacaggtttgacaaggcatattatcaatactagttctcgtttattaaatttgcgtcaagtctctaaaactcgcctttctgaaccaggaaccgatctcgtagaaccttcaccaaaacacacacccaacacgtcagacaacgaaggcagtgacacggaggtctgtggtccaaacagtccttctaaacggggaaacagcacaggaataaagttagtgagaaaagagggtggtctggatgacagtgttttcattgcagttaaagaaattggtcgtgatctgtacaggggcttgcctacagaggaaaggatccagaaactagagttcatgttggataagctacagaatgaaattgatcaggagttggaacacaataattcccttgttagagaagaaaaagagacaactgatacaaggaaaaaatcacttctttctgctgccttagctaaatcaggtgaaaggctacaagctctaacacttcttatgattcactacagagcaggcattgaagatatagaaactttagaaagtctgtctttagaccagcactccaaaaaaataagcaagtacacagatgatacagaagaagaccttgataatgaaataagccaactaatagactctcagccattcagcagcatatcagatgacttatttggcccatccgagtctgtgtagcagacaggtctatttaaactttcaaatgaacagggtaaagttgcatctaaagtaccacagatacaaccatgtttaaatcctcgtatgcactctggcctgcttctccagttacttgcttgtgtaagaacaaaaatgagaaaggttgttttccagtaaaaacatgaccagcttaaaaaaaaaaaaaaaaaaa

SEQ ID NO: 8 - protein, 1154 aa, Homo sapiens
MGLLLMILASAVLGSFLTLLAQFFLLYRRQPEPPADEAARAGEGFRYIKPVPGLLLREYLYGGGRDEEPSGAAPEGGATPTAAPETPAPPTRETCYFLNATILFLFRELRDTALTRRWVTKKIKVEFEELLQTKTAGRLLEGLSLRDVFLGETVPFIKTIRLVRPVVPSATGEPDGPEGEALPAACPEELAFEAEVEYNGGFHLAIDVDLVFGKSAYLFVKLSRVVGRLRLVFTRVPFTHWFFSFVEDPLIDFEVRSQFEGRPMPQLTSIIVNQLKKIIKRKHTLPNYKIRFKPFFPYQTLQGFEEDEEHIHIQQWALTEGRLKVTLLECSRLLIFGSYDREANVHCTLELSSSVWEEKQRSSIKTVELIKGNLQSVGLTLRLVQSTDGYAGHVIIETVAPNSPAAIADLQRGDRLIAIGGVKITSTLQVLKLIKQAGDRVLVYYERPVGQSNQGAVLQDNFGQLEENFLSSSCQSGYEEEAAGLTVDTESRELDSEFEDLASDVRAQNEFKDEAQSLSHSPKRVPTTLSIKPLGAISPVLNRKLAVGSHPLPPKIQSKDGNKPPPLKTSEITDPAQVSKPTQGSAFKPPVPPRPQAKVPLPSADAPNQAEPDVLVEKPEKVVPPPLVDKSAEKQAKNVDAIDDAAAPKQFLAKQEVAKDVTSETSCPTKDSSDDRQTWESSEILYRNKLGKWTRTRASCLFDIEACHRYLNIALWCRDPFKLGGLICLGHVSLKLEDVALGCLATSNTEYLSKLRLEAPSPKAIVTRTALRNLSMQKGFNDKFCYGDITIHFKYLKEGESDHHVVTNVEKEKEPHLVEEVSVLPKEEQFVGQMGLTENKHSFQDTQFQNPTWCDYCKKKVWTKAASQCMFCAYVCHKKCQEKCLAETSVCGATDRRIDRTLKNLRLEGQETLLGLPPRVDAEASKSVNKTTGLTRHIINTSSRLLNLRQVSKTRLSEPGTDLVEPSPKHTPNTSDNEGSDTEVCGPNSPSKRGNSTGIKLVRKEGGLDDSVFIAVKEIGRDLYRGLPTEERIQKLEFMLDKLQNEIDQELEHNNSLVREEKETTDTRKKSLLSAALAKSGERLQALTLLMIHYRAGIEDIETLESLSLDQHSKKISKYTDDTEEDLDNEISQLIDSQPFSSISDDLFGPSESV

SEQ ID NO: 9 - DNA, 3465 bp, Homo sapiens
atggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcaggtttaagccgttttttccataccagaccttgcaaggatttgaagaagatgaagagcatatccatatacaacaatgggcacttactgaaggccgtcttaaagttacgttgttagaatgtagcaggttactcatttttggatcctatgacagagaggcaaatgttcattgcacacttgagttaagcagtagtgtttgggaagaaaaacagaggagttctattaagacggttgaattaataaaaggaaatttacaaagtgttggacttacacttcgtcttgtccagtcaactgatgggtatgctgggcacgtcatcattgaaactgtggctccaaactcgcctgctgcaattgcagatcttcagcggggagatcgacttatcgccattggaggtgtgaaaatcacatcaacactgcaagtgttgaagcttatcaagcaggctggtgaccgagtcctggtgtactatgaaaggcctgttggccagagtaatcaaggtgcagtgctgcaagataactttggccagttggaagaaaactttttgtcaagctcatgccaatcgggttatgaagaggaagctgccgggttgacagtagatactgaaagtagagagctggattctgaatttgaagacttggcaagtgatgtcagagcacaaaatgagttcaaagatgaggcacaatcattaagtcatagtcccaaacgtgttccaacaacactttctattaaaccccttggagctatatcaccagttttaaaccgtaaattagctgtaggaagtcacccactaccaccgaaaattcagtccaaagatggaaataaacctccacccctaaaaacttctgagataacagacccagcacaagtgtcaaaaccaacccaaggatctgctttcaaaccacctgtgccaccacgaccacaagcgaaagttcctttgccttccgccgatgctccaaatcaggcagaaccagatgttctcgttgaaaagccagagaaggtggtgccacctcctcttgtagataaatctgctgaaaagcaagcaaaaaatgtggatgccatagacgatgcagctgcacctaagcaatttttagcaaagcaagaagtggccaaagatgtcacttcagaaacttcctgccctactaaggacagttcggacgaccgtcaaacatgggaatcatcagaaattctttatcgtaataagctaggaaaatggacaagaaccagagcatcctgtttgtttgacatagaagcctgtcacaggtacttaaacattgcattgtggtgcagggatcctttcaagttgggaggtctcatctgtttggggcatgttagtttaaaacttgaagatgtggctttaggatgcctagctacatcaaacacggaatacctttccaaattgagactggaagccccctcacctaaggctatagtcactagaaccgcactacgcaatctgagtatgcaaaagggattcaatgacaaattttgctatggtgacattactattcacttcaaatatttgaaagaaggagaatcagaccaccatgtagttactaacgtagaaaaagaaaaagaaccccatttggttgaagaagtttctgttctccctaaagaggagcaatttgttggacagatgggtttaacagaaaacaaacacagttttcaggatactcagttccagaacccaacatggtgtgactactgtaagaaaaaagtttggactaaagcagcttcccagtgtatgttttgtgcttatgtttgccataaaaaatgtcaagaaaagtgtctagctgagacttctgtttgtggagcaactgataggcgaatagacaggacactgaaaaaccttaggctggaaggacaggaaaccctcttaggcctgcctcctcgtgttgatgctgaagctagcaagtcagtcaataaaacaacaggtttgacaaggcatattatcaatactagttctcgtttattaaatttgcgtcaagtctctaaaactcgcctttctgaaccaggaaccgatctcgtagaaccttcaccaaaacacacacccaacacgtcagacaacgaaggcagtgacacggaggtctgtggtccaaacagtccttctaaacggggaaacagcacaggaataaagttagtgagaaaagagggtggtctggatgacagtgttttcattgcagttaaagaaattggtcgtgatctgtacaggggcttgcctacagaggaaaggatccagaaactagagttcatgttggataagctacagaatgaaattgatcaggagttggaacacaataattcccttgttagagaagaaaaagagacaactgatacaaggaaaaaatcacttctttctgctgccttagctaaatcaggtgaaaggctacaagctctaacacttcttatgattcactacagagcaggcattgaagatatagaaactttagaaagtctgtctttagaccagcactccaaaaaaataagcaagtacacagatgatacagaagaagaccttgataatgaaataagccaactaatagactctcagccattcagcagcatatcagatgacttatttggcccatccgagtctgtgtag

(19) United States
(12) Patent Application Publication
(10) Pub. No.: US 2003/0108915 A1
(43) Pub. Date: Jun. 12, 2003

(54) Glioblastoma multiforme associated protein GliTEN

(75) Inventor: Randall D. McKinnon, Piscataway, NJ (US)

Correspondence Address: PERKINS COIE LLP, POST OFFICE BOX 1208, SEATTLE, WA, 98111-1208, US

(73) Assignee: University of Medicine & Dentistry of New Jersey

(21) Appl. No.: 10/224,624

(22) Filed: Aug. 20, 2002

Related U.S. Application Data
(63) Continuation-in-part of application No. 10/051,769, filed on Oct. 20, 2001.
(60) Provisional application No. 60/242,160, filed on Oct. 20, 2000.

(51) Int. Cl. C12Q 1/68; G01N 33/574; C07H 21/04; C12N 9/00; C12P 21/02; C12N 5/06

(52) U.S. Cl. 435/6; 435/7.23; 435/69.1; 435/320.1; 435/325; 536/23.2; 435/183

(57) ABSTRACT

Nucleic acid sequences that identify a gene product associated with Glioblastoma Multiforme are disclosed. Nucleic acid probes for mRNA transcripts whose expression is associated with glioblast transformation and methods for using these probes in identifying and treating patients at risk for progression into a malignant phenotype are also disclosed.

### CROSS-REFERENCE TO RELATED APPLICATIONS

This application is a continuation-in-part application of U.S. patent application Ser. No. 10/051,769, filed Oct. 20, 2001, which claims priority to provisional patent application U.S. application Ser. No. 60/242,160 (McKinnon, R. D.), filed Oct. 20, 2000, the disclosures of which are incorporated by reference in their entirety herein.

### GOVERNMENT INTEREST

This work was supported by National Institutes of Health Grant RO1 MH54652. This invention was made with government support. The government may own certain rights in the present invention.

### FIELD OF THE INVENTION

The present invention relates to the field of brain cancer therapy, treatment and diagnosis.

Introduction

Glioblastoma multiforme (GBM), the single most fatal form of cancer known to man, has been termed “The Terminator” (Proc.Natl.Acad.Sci 97:6242-44). It is 95% fatal within 10 months of diagnosis, independent of intervention approaches, and there is a disturbing recent increase in incidence especially in the elderly. The disease amounts a terrible toll on patients, families, and clinicians charged with their care. In spite of immense scrutiny, essentially nothing is known of the etiology, cell physiology and molecular genetics of the disease. In addition, attempts at treating the disease have been unsuccessful due to the complex character of the tumor. Thus, novel therapies and treatments for this disease are important and urgently desired.

Several genetic loci are frequently deleted in GBM tumor cells, implicating gene product(s) whose biochemical actions prevent tumor progression (tumor suppressor genes). One such locus, chromosome 10 band q25, is now completely defined by nucleotide sequence data available in public domain data banks, Accession NT000545 at: (http://www.ncbi.nlm.nih.gov).

A large number of previously described genes as well as uncharacterized, predicted protein encoding regions are located at this chromosomal address. However, specific gene products involved in progression of glioblasts into a malignant phenotype have not been elucidated. Identification of specific gene products responsible for tumor progression, and their function in tumor biology, will provide valuable tools in the cancer treatment, therapy and diagnosis.

### SUMMARY OF THE INVENTION

The present invention identifies the exact nucleotide location of a specific gene encoded in locus 10q25 whose expression is altered during progression from normal glioblasts into immortal glial cells, precursors of a malignant phenotype. The present invention is also based, in part, on the discovery of the nucleic acid sequence of the gene, herein termed GliTEN, and its encoded protein. The term GliTEN refers to this genetic locus and is so named to define a novel gene expressed in Glial cells (“Gli”) and encoded within human chromosome 10 (“TEN”). The nucleic acid sequence of the cDNA encoding GliTEN is shown in SEQ ID NO: 7 (FIG. 4). The predicted amino acid sequence encoded by this cDNA is shown in SEQ ID NO: 8.

One aspect of the invention relates to nucleic acid sequences that encode the GliTEN polypeptides described herein. In preferred embodiments, the nucleic acid sequences of the present invention encode the amino acid sequence of SEQ ID NO: 8. In further preferred embodiments, the nucleic acid sequences have the sequence of SEQ ID NO: 7 or SEQ ID NO: 9. The invention further provides for nucleic acid sequences that are the complement of the sequence provided herein. The invention further provides for nucleic acid sequences that hybridize, under stringent hybridization conditions (30 millimolar sodium chloride, 3 millimolar sodium citrate, 0.2% sodium dodecyl sulfate, 68° C.), to the sequences provided herein. In a related aspect, the invention provides method for producing the GliTEN nucleic acid sequences provided herein.

In a related aspect, the invention provides for vectors that include the GliTEN nucleic acid sequences provided herein. In further aspects, the vector(s) is placed in a host cell for production of the GliTEN protein or polypeptide.

Another aspect of the invention provides for GliTEN polypeptides, proteins, fragments or variants thereof and methods for producing the same.

The present invention further relates to an expressed sequence tag (EST), SEQ ID NO: 2, representing the GliTEN gene product associated with immortal glioblasts and GBM. In a further aspect of the invention, methods for using the EST and fragments of the nucleic acid sequence of GliTEN as molecular markers for tumor cell identification and classification are disclosed. Methods for detecting whether a sample from a patient has a propensity for the malignant phenotype are also provided. In yet a further aspect of the invention, methods for using GliTEN polypeptides for therapeutic intervention in brain cancer, including glioblastoma multiforme are disclosed.

An additional aspect of the invention relates to kits for use in diagnosing or identifying candidates at risk for progression into a malignant glioma phenotype.

In yet another aspect of the invention, antibodies capable of recognizing GliTEN polypeptides and methods for producing the same are disclosed. The antibodies may be used for detecting GliTEN in a sample or monitoring the level of GliTEN in a patient.

### BRIEF DESCRIPTION OF THE DRAWINGS

FIG. 1. Partial nucleotide sequence of the region of human chromosome 10 encoding GliTEN (SEQ ID NO: 1). The nucleotide sequence is from the NCBI Genbank data files (accession number AC005887). Shown are regions of the human locus identified by homology to a rodent glioblast-derived EST (clone 24.53, SEQ ID NO: 4), 87% identical to the underlined sequence (SEQ ID NO: 2), and the flanking human sequences encoding an open reading frame (capitalized letters, SEQ ID NO: 3). Double underline: stop codon predicted to lie within intervening (intron) sequences. The encoded protein has high homology (33% and 30% identical, respectively) to the amino (N)-terminus of proteins predicted from genome sequence analysis of Drosophila and C. elegans. All three predicted GliTEN proteins (human, fly and worm) encode an amino terminal “PDZ” domain and a carboxy terminus “C1” domain; these regions of the fly and worm genes encode domains that are highly related (50% amino acid identity) to human chromosome 10 sequences located proximal to the sequence shown.

FIG. 2. Northern blot analysis of GliTEN transcripts in adult rat tissues. Northern analysis of RNA transcripts in adult rat tissues hybridizing to a [32]-P labeled GliTEN cDNA probe (SEQ ID NO: 4). Autoradiographic exposure reveals two distinct transcripts, approximately 7,000 and 4,000 nucleotides in length, present in three independent clones of rat glioblasts (clones i, ii, iii), present at lower levels in adult rat brain and thymus, and present in abundant levels in rat liver. The same transcript was expressed at high levels in a rat kidney cell line (NRK). Equal amounts of poly (A+) selected RNA (1 μg mRNA) from each tissue sample were present on the respective lanes of the nylon membrane, and the exposure time was 16 hours at −70° C.

FIG. 3. Relationship of clone 24.53 (SEQ ID NO: 4) to the full length GliTEN transcript. Left: schematic representation of the GliTEN transcript (a) and associated ESTs including (b) our original EST submission (SEQ ID NO: 2; U.S. patent application Ser. No. 60/242,160 filed Oct. 20, 2000), which is 87% identical to our rodent EST clone 24.53 (SEQ ID NO: 4), (c) Genbank accession BC012186, and (d) Genbank accession AL122051 (clone DKFZp434B0328). Horizontal bars depict relative nucleotide lengths of the respective ESTs. SEQ ID NO: 2 corresponds to nucleotide positions 543-807 of BC012186, and AL122051 overlaps the 3′-terminus of BC012186 (100% identity) from positions 2101-2616. The compiled (annotated) 3,833 nucleotide GliTEN transcript (SEQ ID NO: 7) encodes a single open reading frame (shaded in ‘a’). Right: Polymerase Chain Reaction amplification of human placenta cDNA. PCR products corresponding to the portions of GliTEN are represented in the left schematic, with numbers depicting the location of the 20 nucleotide (nt) PCR primers on the GliTEN transcript (as defined in FIG. 4). Lanes (1-3,5-7): PCR products with 5′ primers unique to BC012186 segment, and 3′ primers unique to AL122051 segment, demonstrating these two separate ESTs are part of a single (GliTEN) transcript. Lane 9: molecular size marker; lane 10: cyclophilin (660 nt) control.

FIG. 4. Annotated nucleotide sequence of GliTEN transcript. Sequences shown are compiled from NCBI Genbank files BC012186 (nucleotide positions 1-2664) and AL122051 (nucleotide positions 2068-3833), with 100% identity in the overlap region (nucleotide positions 2101-2616). Sequences identical to our original EST, clone 24.53, are underlined (nucleotide positions 543-803). Oligonucleotide primer sequences used for the PCR analysis shown in FIG. 3 are underlined and numbered at the right side of the figure.

### DETAILED DESCRIPTION OF THE INVENTION

The chromosome 10 band q25 locus is frequently rearranged and often deleted in brain tumor cells. The deletion of this locus in tumor cells implies that at least some of the genes encoded in this locus are tumor suppressor genes. The 10q25 locus contains a large number of genes and predicted protein encoding regions which have not been characterized. More specifically, defined genes and gene products involved in the progression of glioblasts into a malignant phenotype have not been disclosed. The present invention identifies the location of a gene, referred to as the GliTEN gene, in the 10q25 locus that herein is implicated in progression of brain cells into a malignant phenotype.

The present invention relates to the identification and characterization of novel nucleotide sequences related to the GliTEN gene and novel GliTEN proteins that are encoded by these nucleotide sequences. The invention further relates to methods for preparing the nucleotide and amino acid sequences of the present invention and uses for these sequences.

GliTEN appears to be linked to the progression of brain cells into a malignant cancerous phenotype based on the following: (1) its deletion from chromosome 10q25 in glioblastoma multiforme cells; (2) its increased expression during primary glial progenitor cell transformation; (3) its structural features (PDZ domain) consistent with a biochemical function in regulation of signal transduction pathways; (4) its structural features (C1 domain) consistent with a biochemical function in promoting tumor progression.

One embodiment of the invention provides for nucleic acid sequences that encode the GliTEN polypeptides described herein, wherein the GliTEN polypeptide includes a full length GliTEN protein or fragment thereof, and wherein the fragment includes a biologically active portion of the GliTEN -protein. In one aspect, the invention provides for an isolated nucleic acid sequence, GliTEN (SEQ ID NO: 7), which encodes GliTEN protein (SEQ ID NO: 8). GliTEN (SEQ ID NO: 7) encodes a predicted 1,154 amino acid trans-membrane protein (SEQ ID NO: 8) with conserved domains similar to “scaffolding” proteins involved in growth factor receptor-mediated signal transduction. The domain structure of GliTEN (SEQ ID NO: 8) was determined using the Simple Modular Architecture Research Tool (SMART) analysis software [http://smart.embl-heidelberg.de]. GliTEN includes an amino-terminus hydrophobic “TM” domain characteristic of single span trans-membrane proteins. GliTEN encodes a central “PDZ” domain, a conserved domain found in diverse signaling proteins in bacteria, yeast, plants, insects and vertebrates and thought to function in targeting signaling molecules to sub-membranous sites. GliTEN also encodes a carboxyl-terminus cysteine-rich “C1” (Protein kinase C conserved region 1) domain, characteristic of a family of serine/threonine protein kinases collectively known as protein kinase C (PKC). The C1 region contains a cysteine-rich domain that is essential for binding diacylglycerol (DAG) and phorbol esters (PE), potent tumor promoters that directly activate PKC. All three domains (TM, PDZ, Cl) are conserved in coding sequences of orthologous GliTEN genes encoded in Homo Sapien, Drosophila melanogaster and C. elegans genomes.

Another embodiment provides for isolated nucleic acid sequences selected from a group consisting of: (a) a nucleic acid sequence, SEQ ID NO: 9, comprising nucleotides 178 to 3642 of SEQ ID NO: 7 (the “sense strand” coding region of the GliTEN gene); (b) an “antisense strand” DNA sequence complementary to SEQ ID NO: 9; (c) a “sense strand” RNA sequence equivalent to the sequence listed in (a) and complementary to the sequence listed in (b); (d) an “antisense strand” RNA sequence equivalent to the sequence listed in (b) and complementary to the sequence listed in (a); (e) an oligonucleotide sequence of at least 15 consecutive nucleotides capable of hybridizing to a nucleotide sequence of SEQ ID NO: 9 (a or c, above); and (f) an oligonucleotide sequence capable of hybridizing to a nucleotide sequence of (b or d, above).

Nucleic acid sequences that encode these polypeptides or proteins are collectively referred to as ‘nucleic acids of the invention’ or ‘GliTEN nucleic acids.’ The nucleic acid sequences or molecules of this invention include DNA and RNA molecules, such as genomic DNA, cDNA, and mRNA, and analogs or variants of the DNA or RNA produced. The nucleic acid sequences may be single or double stranded.

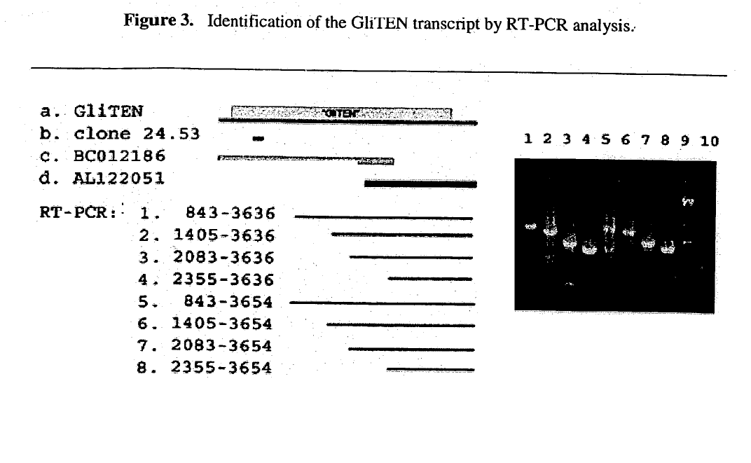

Due to the degeneracy of the genetic code, a number of nucleic acid sequences that encode the GliTEN protein product may be produced. A number of these sequences will only have minimal homology to naturally occurring GliTEN nucleic acid sequence. Each nucleic acid sequence variation based on the various possible codon choices for an amino acid of the GliTEN protein is contemplated by this invention. As such, in addition to SEQ ID NO: 7 and SEQ ID NO: 9 (the coding region of SEQ ID NO: 7), the GliTEN gene refers to any DNA sequence that encodes the GliTEN protein, SEQ ID NO: 8, and any DNA sequence that hybridizes to these sequences and encodes a gene product that is the complement of or functionally equivalent to the GliTEN protein. The hybridization conditions are considered stringent hybridization (nucleic acids that retain binding to GliTEN sequences at a temperature of 68° C. in the presence of 30 millimolar sodium chloride, 3 millimolar sodium citrate, 0.2% sodium dodecyl sulfate). With respect to single-stranded nucleic acid molecules, the term “specifically hybridizing” refers to the association between two single-stranded nucleic acid molecules of sufficiently complementary sequence to permit such hybridization under pre-determined conditions generally used in the art (“substantial complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence. In related embodiments, the antisense GliTEN nucleic acid sequences may be used for regulation of GliTEN gene expression or as antisense primers for use in amplification of GliTEN gene nucleic acid sequences as demonstrated in FIG. 3.

In another embodiment, the GliTEN nucleic acid sequences of this invention include sequences that are at least 70%, 75%, 80%, 85%, 90%, 91%, 92%, 93%, 94%, 95%, 96%, 97%, 98%, 99% or more identical to SEQ ID NO: 7 or SEQ ID NO: 9.

The GliTEN nucleic acid sequences of the present invention may be prepared by two general methods: (1) they may be synthesized from appropriate nucleotide triphosphates, or (2) they may be isolated from biological sources. Both methods utilize protocols well known in the art. The availability of nucleotide sequence information, such as the cDNA having SEQ ID NO: 7 or SEQ ID NO: 9, enables preparation of an isolated nucleic acid molecule of the invention by oligonucleotide synthesis.

In another embodiment, GliTEN nucleic acid sequences and/or their complements are inserted into a vector for amplification of the GliTEN gene using standard recombinant DNA and molecular genetic procedures. In a related embodiment, the vector is placed into a host cell to produce GliTEN protein.

Another embodiment provides for the polypeptide products of the GliTEN nucleic acid sequences. The GliTEN polypeptides, proteins, fragments thereof, and derivatives and other variants of the sequence in SEQ ID NO: 8 thereof are collectively referred to as ‘polypeptides or proteins of the invention’ or ‘GliTEN polypeptides or proteins.’ For example, SEQ ID NO: 7 provides for a predicted GliTEN protein product, SEQ ID NO: 8. In addition, polypeptides encoded by the GliTEN nucleic acid sequences that are substantially the same as the GliTEN protein are provided for.

In a preferred embodiment, the GliTEN protein has an amino acid sequence shown in SEQ ID NO: 8. In other embodiments, the GliTEN protein is substantially the same as SEQ ID NO: 8 and retains the functional activity of the protein of SEQ ID NO: 8. Accordingly, in another embodiment, the GliTEN protein is a protein that includes an amino acid sequence with an overall identity of at least 30% or more (35%, 40% 45%, 50%, 55%, 60%, 65%, 70% 75%, 80%, 85%, 90%, 93%, 95%, 98%) to SEQ ID NO: 8.

Fragments of the GliTEN polypeptides are also included in the invention. A fragment is a polypeptide having an amino acid sequence that entirely is the same as part, but not all, of the amino acid sequence of the aforementioned GliTEN polypeptides. Preferred fragments include, for example, truncation polypeptides having the amino acid sequence of GliTEN polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate GliTEN activity, including those with a similar activity or an improved activity, or with a decreased undesirable activity. Also included are those that are antigenic or immunogenic in an animal, especially in a human.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical.

A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g.., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). As described herein, a “non-essential” amino acid residue is a residue that can be altered from the wild-type sequence of GliTEN without abolishing or more preferably, without substantially altering a biological activity of GliTEN protein. A change in an “essential” amino acid residue abolishes or results in such a change of the GliTEN functional activity.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “identity” or “percent identical” refers to the percent of the amino acids of the subject amino acid sequence that have been matched to identical amino acids in the compared amino acid sequence by a sequence analysis program. “Percent similar” refers to the -percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. H 9:205). When referring to nucleic acid-molecules, “percent identical” refers to the percent of the nucleotides of the subject nucleic acid sequence that have been matched to identical nucleotides by a sequence analysis program.

“Identity” and “similarity” can be readily calculated by known methods. Nucleic acid sequences and amino acid sequences can be compared using computer programs that align the similar sequences of the nucleic or amino acids thus define the differences. In preferred methodologies, the-BLAST programs (NCBI) and parameters used therein are employed, and the DNAstar system (Madison, Wis.) is used to align sequence fragments of genomic DNA sequences. However, equivalent alignments and similarity/identity assessments can be obtained through the use of any standard alignment software. For instance, the GCG Wisconsin Package version 9. 1, available from the Genetics Computer Group in Madison, Wis., and the default parameters used (gap creation penalty=12, gap extension penalty=4) by that program may also be used to compare sequence identity and similarity.

As used herein, “GliTEN activity”, “biological activity of GliTEN” or “functional activity of GliTEN”, refers to an activity exerted by a GliTEN protein, polypeptide or nucleic acid molecule on e.g., a GliTEN-responsive cell or on a GliTEN substrate, e.g., a protein substrate, as determined in vivo or in vitro. The GliTEN activity may be a direct activity, e.g., interacting directly with a target molecule, or an indirect activity. e.g., a cellular signaling activity -mediated by-interaction with other molecules

The present invention also relates to an EST (SEQ ID NO: 2), expressed nucleotide sequence tag, of the GliTEN nucleic acid sequence (SEQ ID NO: 7). Related embodiments of the invention relate to using the EST as a molecular marker for tumor cell identification and classification, and as a target for therapeutic intervention in glioblastoma multiforme. The present invention further relates to fragments of any of the GliTEN gene nucleic acid sequences disclosed herein which may be used in a similar fashion to SEQ ID NO: 2.

One embodiment of the invention relates to an EST comprising the sequence disclosed at SEQ ID NO: 2 which is located at nucleotides 543 to 803 of SEQ ID NO: 7.

In another embodiment, the present invention provides for a method of detecting the presence of GliTEN in a biological sample. The method involves the steps of selecting a probe from SEQ ID NO: 7 which hybridizes to the GliTEN gene, exposing the probe to a biological sample, determining whether the probe hybridizes with the nucleic acid from the sample wherein hybridization of the probe to the sample indicates that GliTEN is present in the sample. In a further embodiment, methods for detecting increased levels of expression of GliTEN are provided. The term “increased levels” relates to the steady-state expression of a nucleic acid sequence or encoded protein in a tumor cell with a higher level, preferably at least two-fold higher, than the level observed in a non-tumor cell from normal tissue (control sample or cell). The above described methods for detecting the presence of GliTEN are employed with the further step of comparing the results to control samples.

In a further embodiment, the present invention provides for primers which are specific for GliTEN. Using PCR techniques, the primers may be employed to amplify nucleic acid sequences and thus detect the presence of GliTEN in a sample.

The present invention also provides for methods of producing the GliTEN polypeptide or fragments of the polypeptide. These methods involve culturing cells which have been exposed to a vector that includes the GliTEN nucleic acid sequence or fragments thereof. The polypeptides may be purified from the host cell or host cell culture. The protocols for the production and purification of recombinant proteins include but are not limited to approaches such as affinity column isolation of GliTEN molecules engineered to express a polypeptide sequence “epitope tag”, such as a histidine tag, using commercial vectors such as the BD Biosciences [http://www.clontech.com/products/catalog02/HTML/1117.shtml] Clontech “Creator” expression system.

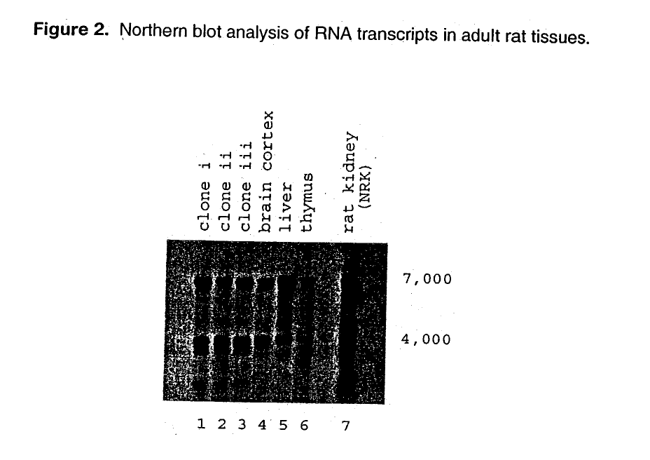

In another embodiment, the present invention is used in the diagnosis of brain cancer. In a preferred embodiment, the present invention is used to diagnose or identify candidates at risk for progression into glioblastoma multiforme. As demonstrated herein, glioblasts express increased levels of nucleic acid associated with SEQ ID NO: 7, SEQ ID NO: 9 and fragments thereof, including SEQ ID NO: 2. Increased levels of these nucleic acids act as a signal to indicate a candidate's risk for progression into the malignant phenotype. The term “increased levels” relates to the steady-state expression of a nucleic acid sequence or encoded protein in a tumor cell with a higher level, preferably at least two-fold higher, than the level observed in a non-tumor cell from normal tissue (control sample or cell). For example, compare FIG. 2 lanes 1, 2, and 3 (immortal glioblasts) with lane 4 (normal adult brain sample).

Hybridization of nucleic acids is typically performed under stringent conditions. The term “stringent conditions” refers to conditions which permit binding of a nucleic acid probe molecule to a highly homologous sequence, and not to non-related sequences, as defined in FIG. 5 of McKinnon et. al., Mol.Cell.Biol. 7:2148-2154, 1987.

The methods for diagnosing or identifying a candidate or patient at risk for progression into the malignant phenotype involve detecting increased levels of SEQ ID NO: 7, SEQ ID NO: 9 and fragments thereof, including SEQ ID NO: 2, expression in a sample from a candidate or patient. An example of a relevant sample would be biopsy material from a patient who has a suspected brain tumor such as low grade astrocytoma or oligodendroglioma, which may have the potential in the absence of aggressive therapy to progress into glioblastoma. Methods for detecting increased levels of nucleic acid expression are well known in the art and can include, but are not limited to, nucleic acid hybridization assays such as Northern blot assay, dot blot assay, microarray assays, in situ hybridization assay, polymerase chain reaction and numerous other techniques and assays or combinations thereof (Sambrook and Russel, Molecular Cloning, A Laboratory Manual, Cold Springs Harbor Laboratory Press, NY, 2001). Labels for use in the detection techniques and assays include, but are no limited to, fluorescent dye molecules, fluorophores such as fluorescein and fluorescein derivatives, radioactive labels, chemiluminescent labels, or enzyme labels. In one embodiment of the invention, probes comprised of fragments of SEQ ID NO: 7, such as SEQ ID NO: 2, are added to a sample which has been obtained from a candidate or patient and attached to a solid support nylon membrane, and the mixture is incubated then rinsed using standard hybridization protocols. The amount of bound probe is quantified using methods including, but not limited to, autoradiographic detection, and compared to a control sample from normal tissue. Increased levels of expression as compared to normal cells is an indication that the candidate or patient is at risk for progression into a malignant phenotype. The probes of the present invention may be prepared using methods which are known in the art, including those methods disclosed in Molecular Cloning, (Sambrook, et al., Eds.).

Another -embodiment of the invention provides for kits for use in diagnosing and/or identifying candidates at risk for progression into the malignant phenotype. The kits comprise probes specific for GliTEN associated nucleic acids. Preferably, the probes comprise fragments of nucleotide sequences SEQ ID NO: 7 and SEQ ID NO: 9, including SEQ ID NO: 2. The kits further comprise reagents and components necessary to perform assays or instructions to practice the methods of this invention.

Another embodiment of the invention provides for kits to use in diagnosing and/or identifying candidates at risk for progression into malignant phenotype. The kits comprise synthetic oligonucleotide probes specific for SEQ ID NO: 7, SEQ ID NO: 9, SEQ ID NO: 2 and GliTEN associated nucleic acids including, aaggtggagttcgaggagctgc (SEQ ID NO: 5), and gtggaagccgccgttgtactcc (SEQ ID NO: 6). The kit further comprises reagents and components necessary to utilize SEQ ID NO: 5 and SEQ ID NO: 6 as primers for polymerase chain reaction (PCR) amplification reaction under standard PCR conditions, to detect the presence and abundance of SEQ ID NO: 7 and SEQ ID NO: 2 in RNA isolated from patient material.

In another embodiment, the present invention relates to antibodies capable of recognizing the GliTEN polypeptide. The antibodies may be monoclonal or polyclonal antibodies. These antibodies may be used for detection of the GliTEN gene product in a biological sample. They may also be used for controlling or monitoring the level of GliTEN gene product and/or activity in a sample or patient. Methods for producing these antibodies are known in the art and include the raising of polyclonal antisera in mammalian species, such as but not limited to rodents rabbits and goats, and the generation of monoclonal mouse cell lines capable of secreting GliTEN specific antibodies after fusion of mouse lymphocytes to immortalized cell lines. The antigenic peptides used to generate GliTEN antibodies can include synthetic peptides manufactured by standard commercial sources [eg www.ptglab.com], or peptide products produced by expression of recombinant plasmid vectors in mammalian cells as define above [0039]. The antibodies of the invention may also be labeled or a conjugated with another molecule for use in detection or targeting.

“Antibodies” as used herein includes polyclonal and monoclonal antibodies, chimeric, single chain, and humanized antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

The term “specific binding affinity” is meant that the antibody or antibody fragment binds to target compounds with greater affinity than it binds to other compounds under specified conditions. Antibodies or antibody fragments having specific binding affinity to a compound may be used in methods for detecting the presence and/or amount of the compound in a sample by contacting the sample with the antibody or antibody fragment under conditions such that an immunocomplex forms and detecting the presence and/or amount of the compound conjugated to the antibody or antibody fragment.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique that-provides for the production of antibody molecules by continuous cell lines in culture. Monoclonal antibodies may be obtained by methods known to those skilled in the art. See, for example, Kohler, et al., Nature 256:495-497, 1975, and U.S. Pat. No. 4,376,110.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the target compound. The term “antibody fragment” also includes single charge antibodies.

In a preferred embodiment, antibodies are prepared, which react immunospecifically with various epitopes of the GliTEN encoded polypeptides. These above-described antibodies may be employed to isolate or to identify clones expressing the polypeptide or to purify the polypeptides by affinity chromatography. Specific antibodies may be made in vivo using recombinant DNA and methods well known in the art.

Antibodies that are immunologically specific to GliTEN proteins, or specific epitopes thereof, may be utilized in affinity chromatography to isolate the GliTEN protein in order to quantify the protein utilizing techniques such as western blotting and ELISA, or to immuno- precipitate GliTEN from a sample containing a mixture of proteins and other biological materials. The immuno-precipitation of GliTEN is particularly advantageous when utilized to isolate potential binding partners of GliTEN, as described above.

Another embodiment provides for methods of detecting and determining the expression status of GliTEN. These methods may be used to diagnose and/or treat individuals at risk for developing cancer by monitoring the levels of GliTEN expression in a patient. Increased levels of expression of the GliTEN gene may suggest that an individual is at risk for progression into the malignant phenotype of glioblastoma multiforme. The methods will involve detecting the levels of GliTEN mRNA and/or GliTEN protein in a test sample of tissue or cells and comparing these levels to those in normal samples of tissues or cells.

### EXAMPLE 1

### Isolation of Rodent Glioblasts and Immortalization In Vitro

Glioblasts are isolated from the rodent brain and maintained in a defined primary cell culture environment in vitro, using the protocols as described (McCarthy and de Vellis, J. Cell Biol 85: 890-902, 1980; Behar et al., J. Neurosci. Res. 21: 168-180, 1988). Glioblasts are obtained from two day old rat brain samples by isolating the cerebral hemispheres, dissociating the tissue by passage through 25-gague needles, then placing the tissue into a culture medium comprised of minimal essential medium supplemented with fetal bovine serum as described (McKinnon et al., Neuron 5, 603-614, 1990). Glioblasts were separated from these cultures by immunoselection (Id.) and placed into fresh culture medium. Under defined culture conditions composed of minimal essential medium supplemented with growth promoting hormones fibroblast growth factor-2 (FGF2), platelet derived growth factor (PDGF, AA-homodimeric form), and insulin (defined below) these primary glioblasts in long term culture (several months) undergo a spontaneous process of immortalization. The cell culture techniques that facilitate this process are described (Neuron 5, 603-614, 1990; J. Neuroscience Research 31:193-204, 1992).

Isolation and culture of primary rat glioblasts is defined as follows. Part 1 is performed on a lab bench with closed door to room to limit air flow, and Parts 2 & 3 are performed in a standard tissue culture hood.

Part 1

Surgical Procedures for Establishing Mixed Rat Brain Glial Cultures

Postnatal day 2 rat pups are decapitated and pinned nose down on a paper towel on top of styrofoam board using 21g needle, then the skin is soaked with 70% ethanol. To remove the brain, the skin is removed from the skull. Next, using small scissors, the skull bone is cut down through front of cranium from midline towards each eye, and again at caudal cranium towards side of neck, then along the midline. Next the skull is flapped open to reveal the forebrain which is removed using sterile curved forceps and placed in a 100 mm tissue culture dish (Falcon) containing 35 ml MEM Hepes (Gibco Biologicals, Bethesda Md.; the media is at 4° C. at the start of this procedure). The brains are then placed on a dissecting microscope, and using sterile No. 5 forceps the meninges are carefully removed. First, the brain is separated into longitudinal halves, then one half is held in place with one forcep and by pinching the olfactory bulb the meninges are peeled off with a second forcep.

Part 2

Cultures

In a tissue culture hood, the cleaned brains are placed in a 50 ml tube (Falcon) dissociated by passing through 19 g, 21 g needles (3 times each direction) and a 25 g needle (1 time) using a 10 cc syringe. The dissociated tissue is passed through a sterile 70 um mesh (Falcon 2350 Cell Strainer, Beckon Dickenson Labware) into a 50 ml tube then centrifuged at 1,000 rpm for 10 minutes. The cell pellet is then resuspended in DMEM (high glucose) with 10% fetal bovine sera (10% DMEM; final volume of 10 ml per 2 brains) and plated at 10 ml per Falcon 75 cm² tissue culture flasks, then place in 37° C. incubator (10% CO2) for 3 days. Primary brain cultures are refed with fresh media every 3 days with DMEM plus 10% FBS, and the flasks generally are confluent by days 5-7.

Part 3

Purification of Primary Glioblasts (Day 8; 2 Hrs)

When confluent the flasks consist of a monolayer of type 1 astrocytes above which are microglia (large, unattached phase bright cells) and glioblasts (small blue cells attached to astrocytes monolayer). To remove microglia, the flasks are placed on a rotary shaker (Innova 2000, New Brunswick Scientific) at 37° C. and shake at ˜110 rpm for 2 hrs then the media is removed and the remaining cells refed with fresh DMEM plus 10% FBS. To detach glioblasts by mitotic shake-off, the flasks are next placed on the rotary shaker at 110 rpm for 12-16 hrs and loose cells collected from the media; glioblasts cells can be harvested 2-3 times for each surgical preparation, and the degree of microglial contamination decreases with each round of purification. The media is centrifuged (1,000 rpm, 10 min) and cell pellets are resuspended in 1.0 ml MEM-Hepes, 0.5% FBS. Glioblasts are further purified by one of several techniques as follows.

[A] Glioblasts can be purified by differential adherence. First, the cells are plated in 10 ml of culture media on a 10 cm Falcon culture dish, then incubate for 30 min at 37° C. After the microglia adhere, the less adherent glioblasts are recovered by gently swirling to suspend the loose cells. [B] Glioblasts can be further purified by removing contaminating microglia by indirect immunopanning. First, cells are incubated (room temp, 10-15 min) with monoclonal antibody A2B5 (1:100 dilution of ascites fluid or 1:10 dilution of tissue culture supernatant) sterilized by filtering through 0.45 uM Costar Spin-X Centrifuge filter units. After incubation, the cells are diluted in 10 mL of 1.0 ml MEM-Hepes, 0.5% FBS then plated on 100 mm Falcon dish and incubate at room temp with no vibrations. After exactly 7 min, the plate is swirled on the lab bench exactly seven times to resuspend non-attached cells which are then harvested from the media by centrifugation as above. Generally 5×10⁶ pure glioblasts are recovered per 15 flasks or 30 animals. [C] Finally, glioblasts are selectively amplified by culturing the cells in the presence of mitogens (10 ng/ml PDGF-AA, 5 ng/ml bFGF) which selectivly amplify glioblast progenitors.

Cells recovered in step [A] or [B] above are recovered by centrifugation, resuspend at 2×10⁶ cells per ml in 10% DMEM, then plated on poly-ornathine coated coverslips or dishes. For coverslips, 25 ul (5×10⁴ cells) are drop seeded onto 12 mm coverslips (generally 10 coverslips per 60 mm dish); for dishes, 1.0 ml (2×10⁶ cells) is placed onto the center of a 60 mm Falcon dish (drop should cover ˜50% of surface area) then the dish carefully into a CO₂ incubator. After 30-60 minutes the cells attach, and 10% DMEM is added to a total of 5 ml per 60 mm dish (10 ml per 10 mm dish) and left at 37° C. for at least 12 hrs. On day 2 (15-24 hrs. later), the media is replaced with defined media consisting of Gibco/BRL Dubelcco's MEM, high glucose plus 1 mm Na pyruvate, 25 ug/ml gentamicin, 0.5% FBS, 50 ug/ml transferrin, 25 nM selenium, 30 nM T3, 50 ng/ml bovine insulin.

REAGENTS: (1) Animals: Sprague Dawley rat pups (with mom), 2 days old on arrival from Taconic Farms, N.Y. (2) Equipment: a) A good dissecting microscope (Zeiss Stemi SV6), a hemocytometer. b) Sterile surgical tools: No. 5 forceps, curved forceps, small & large scissors. c) One each: 12 cc syringe, 19 g, 21 g, 25 g needles. d) Falcon cell strainer (Falcon 2350 Cell Strainer; Beckon Dickenson Labware). e) Spin-X filters (Costar Spin-X Centrifuge filter units, Cat. No. 8162). f) Glass coverslips (Fisher Scientific, 12 mm). (3) Tissue Culture Reagents: a) tissue culture facility, plastic dishes, culture media. b) Fetal bovine sera (Hyclone, Inc.) thawed at 4° C. and not heat inactivated. c) Defined media supplements: [transferrin: Sigma #T2252; 10 mg/ml in PBS, freeze; selenium: Sigma #59133; 3 mM, freeze; tri-ido thyronine (T3): Biofluids #354; 30 mM, freeze; bovine insulin: Sigma #T1882; 10 mg/ml, 4° C., 4.01N HCL]. d) Poly-L-Lysine (Sigma). (4) Bench top centrifuge (50 ml tubes, 1,000 rpm). (5) Antibodies: supernatant fluids obtained from monoclonal producer cell lines A2B5, 04 (American Tissue Culture Collection, Rockville Md.), (6) Growth Factors (Upstate Biotech, Inc.): Basic fibroblast growth factor (human recombinant; stock=1 ug/ml, final=1-5 ng/ml; Platelet derived growth factor (PDGF) human recombinant PDGF-AA, stock=10 ug/ml, final=10 ng/ml.

### EXAMPLE 2

### Analysis of Glioblast Transformation

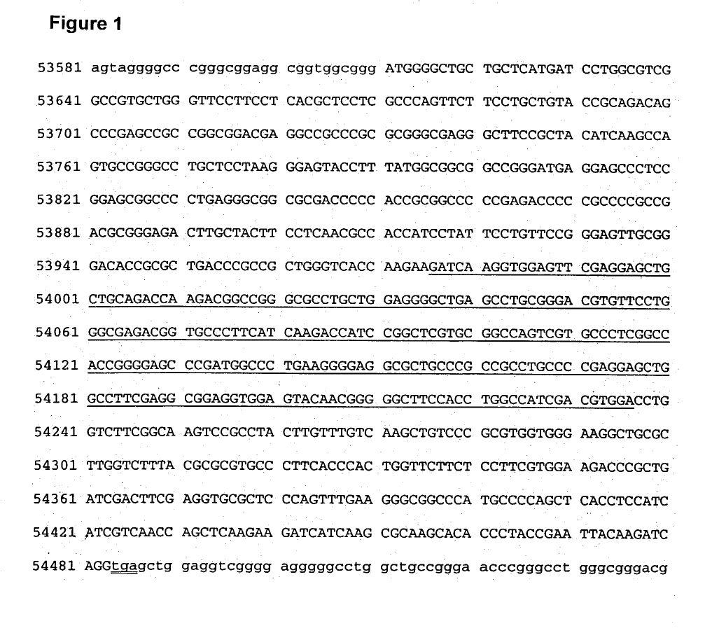

The biological process of glioblast immortalization results in the focal growth of primary cells that no longer require mitogens (eg recombinant growth factors) to sustain their proliferation in vitro. The molecular processes underlying this transformation process was examined, and the resulting array of expressed nucleic acid (RNA) transcripts was characterized using the published techniques of subtractive hybridization (Representational Difference Analysis, RDA; Nucleic Acids Res. 22:5640-48, 1992). Several (n=155) expressed sequence tags (ESTs) whose mRNA transcripts were maintained at an elevated steady state level in immortal glioblasts were characterized. The characterized ESTs were examined individually by determining their DNA sequence, using standard approaches in a core sequencing facility. The obtained sequences were imported into NCBI Blast (http://www.ncbi.nlm.nih.gov/BLAST) to screen for potentially related nucleic acid sequences in public domain databases. One EST transcript (clone number 24.53) (SEQ ID NO: 4), represents a glioblast EST that maps to human chromosome 10 band q25 (Genbank accession AC005887) with 86% nucleic acid sequence identity, confirming that the mRNA transcript of cDNA 24.53 is the rat homologue of a human mRNA transcript (FIG. 1). The nucleotide sequence of SEQ ID NO: 4 is gat caaggtggag ttcgaggagc tgctgcagac caagacggcc tttttttttt tggaggggct gagcctgcgc gacgtgttcc tgggtgacac cgtgccctac atcaagacca tccggctggt gcggcccgtg gtggcttcgg gcaccggcga gcccgacgaa cccgatgggg acgctctgcc cgccacctgc ccgggggagc tggcctttga ggcggaggtg gagtacaacg gcggcttcca cctggccatc gacgtggatc,

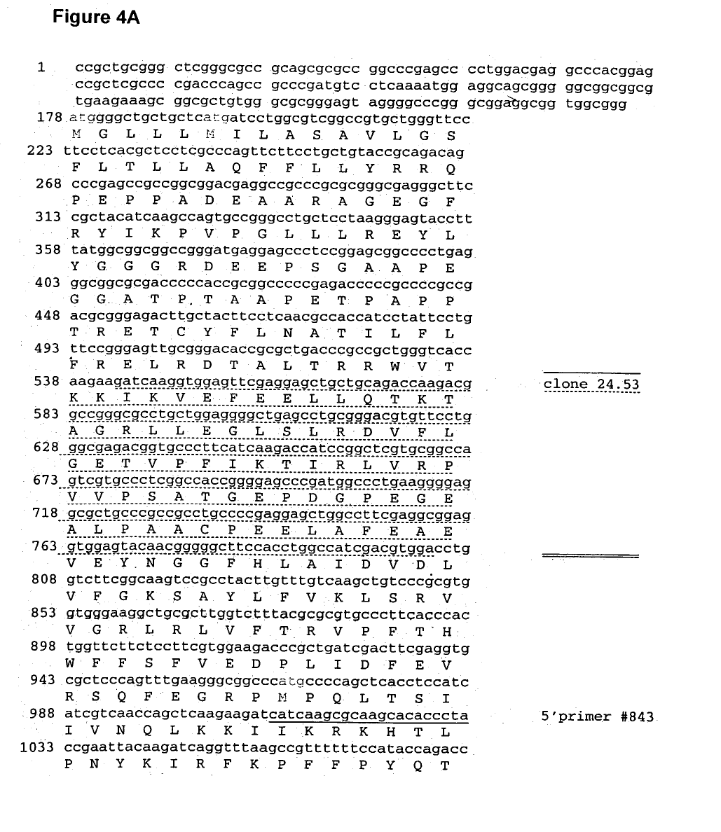

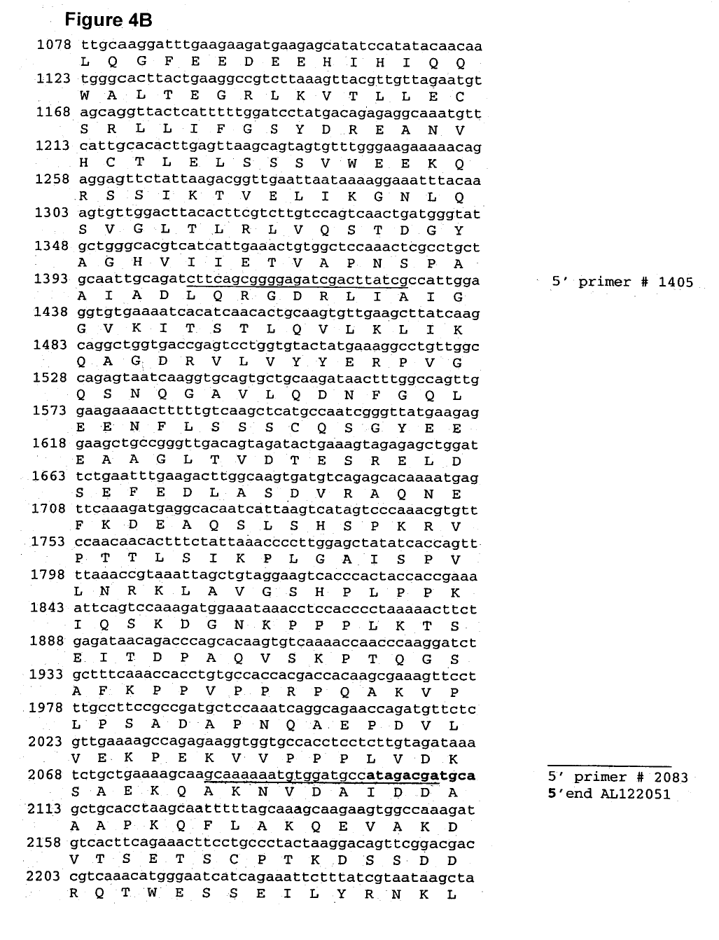

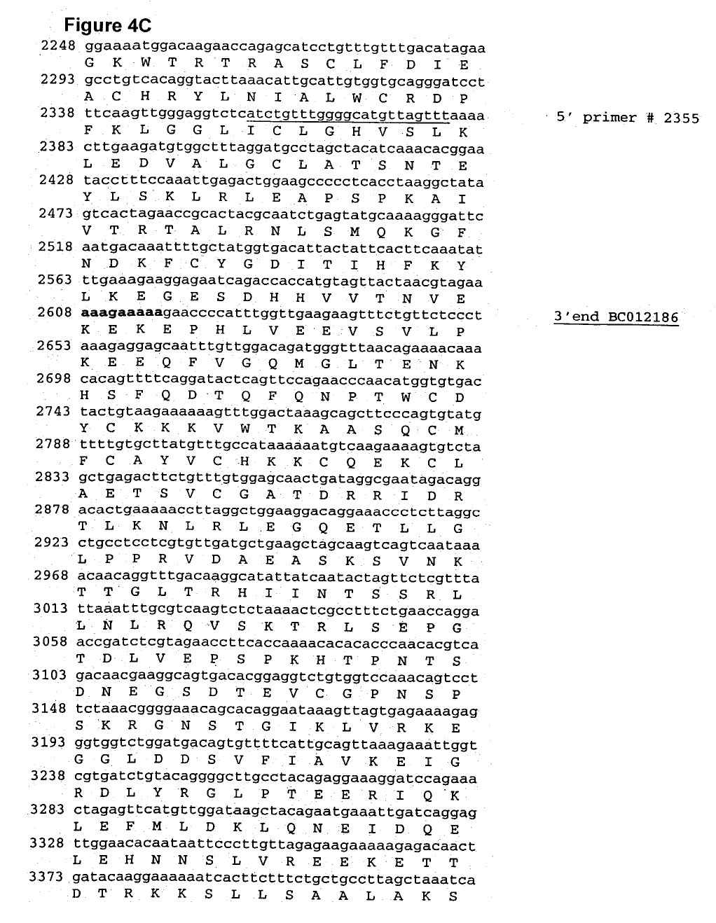

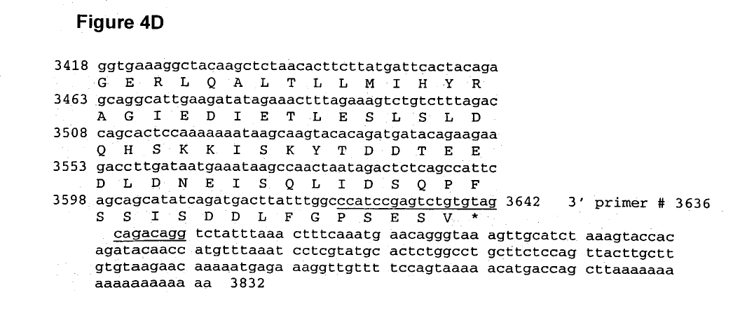

Based on homology of clone 24.53 with a larger EST in the public domain databank (BC012186; FIG. 3), and overlap of BC012186 sequences with a second EST (AL122051; FIG. 3), the nucleotide sequence of the GliTEN transcript was compiled (FIG. 4). The size of this compiled GliTEN transcript is equivalent to that of the smaller of two mRNA species detected with a radiolabeled probe comprised of clone 24.53 sequences (FIG. 2).

To demonstrate that the three ESTs (clone 24.53; BC012186; AL122051) are from a single, contiguous GliTEN mRNA transcript we performed a reverse transcriptase—polymerase chain reaction (RT-PCR) analysis (FIG. 3). RNA samples from several sources, was isolated using TRIzol reagent (Gibco BRL; manufactures protocol) then reverse transcribed into ‘first strand’ cDNA using Moloney MuLV reverse transcriptase (Gibco) under standard conditions. The cDNA was subjected to PCR amplification using synthetic oligodeoxynucleotide primers defined by the predicted GliTEN transcript and identified by nucleotide position on the GliTEN sequence (FIG. 4). Sense strand “upstream” (5-prime) and antisense strand “downstream” (3-prime) primer pairs used for each reaction are identified by their sequence location (FIG. 3, left panel). PCR amplification was performed with upstream primers that identify sequences specific to BC012186 (#843, #1405, #2083) plus downstream primers that are specific to AL122051 (#3636, #3654). In all samples a PCR product of the predicted size ‘bridging’ these two EST sequences was observed (FIG. 3 right panel, lanes 1-3, 5-7). The most consistent conclusion of this observation is that these two ESTs (BC012186; AL122051) represent partial fragments of a single mRNA species, herein defined as GliTEN. The complied GliTEN transcript represents a novel mRNA species which, to date, has not been described in public databanks.

The nucleic acid sequence of GliTEN, SEQ ID NO: 7, contains a single open reading frame (bases 178 to 3642 of SEQ ID NO: 7) the hypothetical protein translation is shown in FIG. 4. The open reading frame is predicted to encode a 1154 amino acid protein with a predicted molecular mass of 114,554 kilodaltons, SEQ ID NO: 8 (see FIG. 4). The predicted protein includes an amino-terminus hydrophobic (predicted transmembrane) domain, a central “PDZ” domain, and a carboxyl-terminus “Cl” domain. These structural features suggest the GliTEN protein may be localized to the inner face of the cell membrane and functions as a ‘scaffolding’ molecule, under regulations similar to Protein Kinase C, and is likely involved in signal transduction processes.

### EXAMPLE 3

### Northern Blot Analysis of GliTEN Transcripts

Total cell RNA was isolated from tissue samples using commercially available reagents and procedures described therein (Gibco Trizol), obtained from animal organs, from animal cells in culture, or from patients at the time of surgical biopsy or tumor resection. Poly (A)-selected mRNA from adult rat tissues were separated by agarose gel electorphoreses, transferred by blotting to a nylon membrane, -and the blot was probed with rat glioblast EST probe 24.53 (FIG. 2). The blot contained 1 μg MRNA from each tissue, and the exposure time was 16 hours at 70° C. Examination of mRNA transcripts revealed hybridization to two transcripts approximately 7,000 and 4,000 nucleotides in length, expressed at high levels in three independently derived immortal glioblast cell lines in vitro and in several adult tissues including brain and liver (FIG. 2). Analysis of CDNA generated from RNA samples by RT-PCR further confirmed the presence of a cognate of this transcript in human brain. For PCR, RNA is reverse transcribed into single stranded cDNA using oligo (dT) primer using commercially available kits (Gibco BRL) for cDNA synthesis and procedures described therein. PCR analysis was performed using 100 ng template cDNA in a 50 ul reaction consisting of 0.25 uM synthetic oligodeoxynucleotide primers (SEQ ID NO: 5, SEQ ID NO: 6), 0.1 mM dNTP's, 2.5 mM MgC12, 5 units Taq polymerase (Gibco/BRL, Bethesda MD) and Taq reaction buffer supplied by the manufacturer. The primers, SEQ ID NO: 5 and SEQ ID NO: 6, for amplification of the SEQ ID NO: 2 (GliTEN transcripts) were obtained from commercial vendors (IDT, Coralville Iowa). PCR amplification was performed using a Perkin-Elmer thermocycler with 30 cycles [95° C., 1 min; 58° C., 2 min; 72° C., 3 min] followed by 10 min at 72° C. extension. The PCR products were separated on 1.5% agarose gels containing 0.5 ug/ml ethidium bromide, and DNA products were visualized by UV trans-illumination. All electrophoretic analysis included a DNA mobility marker (HaeIII digest of psi-X174 DNA, Gibco/BRL), and PCR products were identified by relative electrophoretic mobility.

### EXAMPLE 4

### GliTEN, Gene Product Located on Human Chromosome 10 Band q2.5

Examination of the human 10q25 chromosomal locus (FIG. 1) revealed a 892 base pair (bp) region flanking the original EST with a single open reading frame for protein translation (AC005887, nucleotide positions 53,611-54,483 inclusive). The predicted protein endocing this and flanking sequences (FIG. 4) is herein referred to as GliTEN, since the original rodent EST (clone 24.53) was identified in immortal Glioblasts and since the rodent EST (clone 24.53) maps to human chromosome ten, a locus whose mutation is associated with glioblast transformation.

A sequence alignment search for proteins related to this predicted protein using the NCBI Genbank ‘tblast’ algorithm revealed two highly homologous proteins predicted to be encoded in the genomes of Drosophila melanogaster (CG10362, Genbank accession AAF48119) and C. elegans (Genbank accession CAB54213). The D. melanogaster sequence has been detected as ESTs (clone numbers CK2546, LD34222) expressed in the embryonic brain, as reported by the Berkeley Drosophila Genome Project (http://www.fruitfly.org;). Neither the fly or worm homologue has been further characterized, and to date these molecules are defined only as ‘theoretical’ gene products.

In summary, analysis of transcripts that were elevated in the process of rodent glioblast immortalization led to the identification of an mRNA transcript that had not been previously characterized in any organism. The human homologue of this transcript was then mapped to human chromosome 10q25, which is associated with brain cancer, and a predicted protein, GliTEN, was determined and implicated in the process of glioblast transformation and turmorogenesis.

### EXAMPLE 5

### Methods for Detecting Candidates At Risk for Progression into GBM

The nucleotide sequence herein referred to as EST (SEQ ID NO: 2), encoding a portion of the gene product GliTEN, is a molecular probe for mRNA transcripts whose expression is associated with glioblast transformation. Fragments of SEQ ID NO: 7 and SEQ ID NO: 9 may also be used as probes for GliTEN mRNA transcripts. SEQ ID NO: 2 serves as a probe for characterizing glioblast tumors in humans, with specific emphasis on its use in identification of tumors which are likely candidates for progression into glioblastoma multiforme.

The probe defined herein as SEQ ID NO: 2 represents a molecular marker for determining the abundance of RNA transcripts of this sequence present in normal, immortal, and pre-malignant cells. The abundance of these RNA sequences is determined by methods including but not limited to RNA blot analysis, using SEQ ID NO: 2 as a molecular identifier for the presence of such RNA transcripts, or PCR amplification, using SEQ ID NO: 5 and SEQ ID NO: 6. Samples to be examined by this analysis are obtained from patients by surgical resection, such as but not limited to surgical biopsy material and surgical specimens removed from a patient at the time of surgical resection to debulk an existing tumor.

Samples are immediately processed for the isolation of total cell RNA molecules from this tissue using the Trizol reagent and protocols as detailed by the reagent manufacturer (Gibco BRL), these representing standard protocols for the isolation of total cell RNA from any source of tissue. Blot analysis is defined as the fractionation of a sample of said tissue RNA (5-10 micrograms is generally sufficient) on an agarose gel containing formaldehyde, with adjacent lanes containing appropriate control tissue samples, test samples, and molecular weight markers, as described in McKinnon et al (Neuron 5,603-614,1990).

The samples are then transferred to nylon membranes and processed for hybridization analysis with SEQ ID NO: 2 labeled probe using standard conditions as described in Sambrook and Russel (Molecular Cloning, a laboratory manual, 3rd Edition; Cold Spring Harbor Laboratory Press, 2001). In the case of p³² labeled radioactive probes, the RNA transcripts hybridizing to SEQ ID NO: 2 are visualized, after probing and subsequent washing of the blot to high stringency, by exposing the nylon membrane to an emulsion film (Fuji RX medical X-ray film) and developing the resulting autoradiographic exposure. Control samples include, but are not limited to, RNA isolated from non-cancerous ‘normal’ tissue obtained during the procedure that generated the suspected or known tumor specimen, RNA isolated from human cell lines with characteristics similar to those of the cancerous lesion (human tumor cell lines are commercially available in public repositories such as American Type Culture Collection, Rockville Md.), RNA isolated from normal rat brain glioblasts, and RNA isolated from immortal rat brain glioblasts.

The blot analysis of transcripts expressed in a patient's sample will identify a 4,000 nucleotide RNA containing sequences complementary to the probe SEQ ID NO: 2, that represent the bona fide messenger RNA encoding the GliTEN protein. PCR analysis of SEQ ID NO: 2 expression in such samples would be undertaken after reverse transcription of such RNA samples, and subsequent PCR amplification using the SEQ ID NO: 7 specific primers, such as SEQ ID NO: 5 and SEQ ID NO: 6, as outlined in Example 3 above. The results of this analysis will reveal the level of expression of these specific RNA transcripts in the patient samples, and will allow a determination of their level of expression in those samples relative to normal tissue, non-cancerous tissue, and cancerous tissue.

An elevated level of expression, detected as a specific elevation in the intensity of autoradiographic signal of SEQ ID NO: 7 transcripts,. is observed in immortal rat glioblasts relative to their levels in normal primary culture rat glioblasts. A similar elevated level of SEQ ID NO: 7 transcripts in a surgical biopsy from a suspected brain lesion, relative to the level of SEQ ID NO: 7 transcripts in adjacent normal tissue, is taken as evidence that the suspected lesion site contains cells which have the potential to progress into glioblastoma multiforme. Such evidence gives reasonable grounds for the need to pursue an aggressive clinical strategy to eliminate such lesions from the patient.

### EXAMPLE 6

### Method for Characterizing GliTEN

SEQ ID NO: 2 represents a short segment of a large (4,000 nucleotide) RNA transcript expressed in immortal glioblasts. The protein GliTEN encoded within this sequence, based on homology between human, Drosophila and C.elegans genomic sequences, is predicted to have a molecular size of 114,554 kilodaltons encoded in approximately 3,500 nucleotides of this transcript. The full length CDNA encoding GliTEN is obtained from normal glial cells by selective PCR amplification of the transcript, using standard molecular biological procedures. RNA from tissues containing SEQ ID NO: 2 transcripts is isolated and reverse transcribed into first strand cDNA as described in Example 5, then PCR amplified using sets of oligodeoxynucleotide primers including SEQ ID NO: 5 and SEQ ID NO: 6.

To isolate sequences from the 5′ portion of the molecule, PCR reactions are carried out using a commercial kit (InVitroGen) employing the 5′-RACE protocol. To isolate sequences from the 3′ portion of the molecule, PCR reactions are carried out using SEQ ID NO: 5 and the 3′-primer oligo(dT). PCR products are amplified using standard thermocycling conditions, and the products obtained are identified by direct DNA sequence analysis from a Core sequencing facility. The respective 5′ and 3′ sections of the complete cDNA are assembled in a plasmid vector and amplified using standard bacteriological cloning as described in Sambrook and Russel (Molecular Cloning, a Laboratory Manual, 3rd Edition; Cold Spring Harbor Laboratory Press, 2001).

Based on these findings, it is believed that GliTEN will be useful in therapy and treatment of brain cancers, including GBM, since its delivery into glioblastoma tumor cells may suppress the malignant phenotype in patients. The encoded gene product GliTEN may be used as a tumor suppressor in preventing glioblast transformation, and thus the GliTEN transcript may be used in methods for treating GBM, including gene therapy.

Accordingly, further embodiments of the invention involve vectors for use in cancer treatment, comprising a viral or plasmid vector encoding a promoter linked to a GliTEN expression cassette. In a further embodiment, the vectors of this invention may be used in gene therapy approaches to treat cancer, including glioblastoma multiforme. The gene therapy techniques are employed to increase expression of the GliTEN gene in tumor cells, whereby increased expression of GliTEN may suppress tumor growth. Gene therapy techniques allow an absent gene to be replaced with a functional gene. This invention allows for the replacement of an absent gene, which is believed to encode a tumor suppressor protein located in 10q25, with a functional gene. Gene therapy techniques also allow for the delivery and controlled expression of therapeutic gene products. In a further embodiment, the vector containing the GliTEN expression cassette is delivered to the tumor, such as glioblastoma multiforme. The gene therapy techniques may employ adenoviral vectors, adeno-associated viral vectors, recombination-defective retroviral vectors or plasmid DNA vectors to deliver the GliTEN expression cassette into the tumor or cancerous cells. The vectors of this invention may be used to increase GliTEN levels within tumor cells and thereby suppress tumor growth.

The term “vector” refers to a nucleic acid construct engineered to encode a particular gene product. The vectors of the present invention can include adenoviral, adeno-associated viral, recombination-defective retroviral, or plasmid DNA vectors. The vectors include all necessary sequences for the expression of the GliTEN expression cassette and any sequences that may be included to control the expression of the cassette. These sequences may include, but are not limited to, a promoter or initiation sequence, an enhancer sequence, termination sequence, RNA processing signals, and/or a polyadenylation signal sequence.

The term “GliTEN expression cassette” refers to nucleic acid which codes for the GliTEN protein product as defined in Example 6. Due to the degeneracy of the genetic code, a number of nucleic acid sequences that encode the GliTEN protein product may be produced. A number of these sequences will only have minimal homology to the naturally occurring GliTEN nucleic acid sequence. Each nucleic acid sequence variation based on the various possible codon choices is contemplated by this invention. The expression cassette is positioned within the vector such that it can be transcribed into RNA and translated into the GliTEN protein product.

The term “necessary sequences for the expression of GliTEN” refers to sequences required to ensure the RNA transcription and subsequent translation of the expression cassette to produce GliTEN polypeptide sequences. The term “promoter” refers to a DNA sequence that is bound by RNA polymerase and is required to initiate RNA transcription of a gene. There are a number of promoters that are known in the art, including those that can enhance or control expression of the gene or expression cassette. For example, cytomegalovirus early promoter may be fused to the GliTEN expression cassette to obtain constitutive expression of the cassette.

The vectors of this invention may be delivered directly to the location of the tumor cells by injection. The vectors may be administered or delivered in saline solutions or encapsulated in liposomes. Delivery into the area of the tumor is performed at the time of biopsy or after a surgical debulking procedure. The term “tumor” refers to cancerous cells, including those with a malignant phenotype, such as glioblastoma multiforme.

Methods for increasing the level of GliTEN expression in glioblastoma cells are provided for. These methods involve administering vectors encoding GliTEN polypeptide to glioblastoma cells, wherein expression of the vector increases the level of GliTEN in the cells. The methods also involve administering GliTEN polypeptides to the glioblastoma cells.

Methods for treating glioblastoma multiforme are also disclosed. These methods involve administering vectors encoding GliTEN polypeptide or GliTEN polypeptides to glioblastoma cells.

In related embodiments, methods for decreasing the risk of brain tumor cells entering the malignant stage of glioblastoma multiforme are disclosed. Antibodies specific for the GliTEN polypeptide are administered to the brain tumor cells. The levels of GliTEN in cells may decrease upon binding to the antibodies. In a further related embodiment, candidates at risk for progression into a malignant glioma phenotype are identified and antibodies specific to GliTEN polypeptides are administered to the candidates in order to decreases the levels of GliTEN in the candidate. In a further related embodiment, compounds capable of binding to the C1 or PDZ region of GliTEN are administered to the candidate in place of or in combination with the antibodies specific for GliTEN.

In yet a further related embodiment, a candidate at risk for progression into the malignant phenotype of glioblastoma may minimize the risk by monitoring its levels of GliTEN expression and by administering antibodies specific to GliTEN or compounds capable of binding the C1 or PDZ region of GliTEN when increased levels of GliTEN are detected during monitoring.

Kit for use in the treatment of glioblastoma multiforme are also disclosed. These kits include vectors encoding GliTEN polypeptides or GliTEN polypeptides and instructions for administration. In a related embodiment, kits for use in minimizing the risk of a candidate's progression into the malignant phenotype of glioblastoma are provided for. These kits include nucleotide sequence probes of SEQ ID NO: 2, SEQ ID NO: 5, SEQ ID NO: 6, SEQ ID NO: 7, SEQ ID NO: 9 or fragments thereof, reagents and components for use in performing assays, antibodies specific to GliTEN, and instructions for use.

One skilled in the art will readily appreciate that the present invention is well adapted to carry out the objects and obtain the ends and advantages mentioned as well as those inherent therein. The nucleic acid sequences along with the methods and procedures described herein are presently representative of preferred embodiments and are exemplary and not intended as limitations on the scope of the invention. Changes therein and other uses will occur to those skilled in the art which are encompassed within the spirit of the invention or defined by this scope with the claims.

It will be readily apparent to one skilled in the art that varying substitutions and modifications may be made to the invention disclosed herein without departing from the scope and spirit of the invention.

All patents and publications referenced herein are incorporated by reference to the same extent as if each individual publication was specifically and individually indicated to be incorporated by reference.

What is claimed is:

1. An isolated nucleic acid molecule comprising the nucleotide sequence of SEQ ID NO: 7 or 9.

2. An isolated nucleic acid molecule comprising a sequence that hybridizes under stringent conditions to the isolated nucleic acid molecules of claim 1.

3. An isolated nucleic acid molecule comprising a nucleotide sequence encoding a polypeptide comprising the amino acid sequence of SEQ ID NO: 8.

4. A vector comprising the isolated nucleic acid molecules of claim 1.

5. A vector comprising the isolated nucleic acid molecules of claim 2.

6. A vector comprising the isolated nucleic acid molecules of claim 3.

7. A host cell comprising the vector of claim 4.

8. A host cell comprising the vector of claim 5.

9. A host cell comprising the vector of claim 6.

10. An isolated nucleic acid molecule comprising a nucleotide sequence having at least 70% identity with the isolated nucleic acid molecules of claim 1.

11. An isolated nucleic acid molecule comprising a nucleotide sequence encoding a polypeptide comprising the amino acid having at least 70% identity to SEQ ID NO: 8.

12. An isolated polypeptide comprising the amino acid sequence of SEQ ID NO: 8.

13. An isolated polypeptide comprising an amino acid sequence having at least 70% identity with the isolated polypeptides of claim 12.

14. A probe for use in identifying a patient at risk for progression into the malignant phenotype comprising the nucleotide sequence of SEQ ID NO: 7 or 9, or a fragment or complement thereof, or the labeled nucleotide sequence of SEQ ID NO: 7 or 9, or a fragment or complement thereof.

15. The probe of claim 14, wherein the label is a fluorescent dye molecule, a radioisotope, a chemiluminescent molecule, or an enzyme.

16. A method for detecting whether a patient is at risk for progression into glioblastoma multiforme comprising,
a) providing a sample from a patient;
b) adding a labeled probe comprising a nucleotide sequence of SEQ ID NO: 7 or 9 or a fragment or complement thereof to the sample or performing PCR analysis using SEQ ID NO: 5 and SEQ ID NO: 6;
c) analyzing levels of mRNA bound with the probe;
d) treating a control sample according to the method to assess the level of mRNA in a control sample; wherein the presence of increased levels of mRNA expression in the sample in an amount higher than the control sample indicates risk for progression into glioblastoma multiforme.

17. A kit for use in detecting whether a patient is at risk for progression into glioblastoma multiforme comprising nucleotide sequence probes of SEQ ID NO: 7 or 9 or fragments thereof and instructions for use.

18. The kit of claim 17, further comprising reagents and components for use in performing assays.

19. A kit for use in detecting whether a patient is at risk for progression into glioblastoma multiforme comprising nucleotide sequence probes of SEQ ID NO: 5 and SEQ ID NO: 6 and instructions for use.

20. The kit of claim 19, further comprising reagents and components necessary to use SEQ ID NO: 5 and SEQ ID NO: 6 as primers for PCR amplification reaction.

21. An isolated nucleic acid molecule selected from the group consisting of: (a) SEQ ID NO: 7 or 9; (b) a sequence encoding a polypeptide comprising the sequence of SEQ ID NO: 8; (c) a sequence having 90% identity to the sequences of (a) or (b); (d) a sequence complementary to the sequences of (a)-(c); (e) a sequence capable of hybridizing to the sequences of (a)-(d) under stringent conditions.

22. A vector comprising the nucleic acid molecules of claim 21.

23. A host cell comprising the vector of claim 22.

24. A method for producing a polypeptide comprising the step of culturing the host cells of claim 23.

25. A probe for use in identifying a patient at risk for progression into the malignant phenotype comprising labeled antibodies capable of recognizing the GliTEN polypeptides.

26. The probe of claim 25, wherein the label is a fluorescent dye molecule, a radioisotope, a chemiluminescent molecule, or an enzyme.

27. A method for detecting whether a patient is at risk for progression into glioblastoma multiforme comprising,
a) providing a sample from a patient;
b) adding a labeled probe of claim 25 to the sample;
c) analyzing levels of protein bound with the probe;
d) treating a control sample according to the method to assess the level of protein in a control sample; wherein the presence of increased levels of protein expression in the sample in an amount higher than the control sample indicates risk for progression into glioblastoma multiforme.

28. A method for increasing the level of GliTEN expression in glioblastoma cells comprising the step of administering a vector encoding a GliTEN polypeptide to the glioblastoma cells, wherein expression of the vector increases the level of GliTEN in the cells.

29. A method for increasing the level of GliTEN in glioblastoma cells comprising the step of administering GliTEN polypeptides to the glioblastoma cells.

30. A method for treating glioblastoma multiforme comprising the step of administering a vector encoding a GliTEN polypeptide to glioblastoma cells.

31. A method for treating glioblastoma multiforme comprising the steps of administering GliTEN polypeptide to glioblastoma cells.

32. A method for decreasing the risk of brain tumor cells entering the malignant stage of glioblastoma multiforme comprising the step of administering antibodies specific for the GliTEN polypeptide to the brain tumor cells to decrease levels of GliTEN in cells.

33. A method for decreasing the risk of normal glioblast cells from progressing into immortal glial cells comprising:
identifying a candidate at risk for progression into a malignant glioma phenotype; and
administering antibodies specific to GliTEN polypeptides, wherein the binding of the antibodies to the GliTEN polypeptides decreases the levels of GliTEN in the candidate.

34. A method for decreasing the risk of glioma cell progression into a malignant phenotype comprising the step of administering a compound capable of binding to the C1 region of the GliTEN polypeptide.

35. A method for decreasing the risk of glioma cell progression into a malignant phenotype comprising the step of administering a compound capable of binding to the PDZ region of the GliTEN polypeptide.

36. A method for minimizing the risk of a candidate's progression into the malignant phenotype of glioblastoma comprising:
monitoring levels of GliTEN expression in a candidate; and
administering antibodies specific to GliTEN to the candidate when increased levels of GliTEN are detected during monitoring.

37. A kit for use in the treatment of glioblastoma multiforme comprising:
vectors encoding GliTEN polypeptides or GliTEN polypeptides; and
instructions for administration.

38. A kit for use in minimizing the risk of a candidate's progression into the malignant phenotype of glioblastoma comprising:
nucleotide sequence probes of SEQ ID NO: 2, SEQ ID NO: 5, SEQ ID NO: 6, SEQ ID NO: 7, SEQ ID NO: 9 or fragments thereof;
reagents and components for use in performing assays;
antibodies specific to GliTEN; and
instructions for use.
